(19) United States
(12) Patent Application Publication
(10) Pub. No.: US 2026/0000369 A1
(43) Pub. Date: Jan. 1, 2026

(54) METHODS FOR SELECTING INDIVIDUAL COMPONENTS OF A MEDICAL IMAGING APPARATUS WITH LARGE LANGUAGE MODEL

(71) Applicant: Siemens Healthineers AG, Forchheim (DE)

(72) Inventors: Florian WOLZ, Erlangen (DE); Richard DIRAUF, Erlangen (DE)

(73) Assignee: Siemens Healthineers AG, Forchheim (DE)

(21) Appl. No.: 19/254,640

(22) Filed: Jun. 30, 2025

(30) Foreign Application Priority Data
Jul. 1, 2024 (DE) 10 2024 206 133.6

(51) Int. Cl. A61B 6/03; A61B 6/42; A61B 6/46

(52) U.S. Cl. CPC A61B 6/03; A61B 6/4208; A61B 6/467

(57) ABSTRACT

One or more example embodiments relates to a method for selecting individual components of a medical imaging apparatus by selecting a group of individual components from a component set by way of an algorithm for machine learning. A text input and/or a prompt is provided. The text input is used by the algorithm in the selection of the group of individual components. With the text input, the selection and/or sorting can be specified very easily.

### CROSS-REFERENCE TO RELATED APPLICATION(S)

The present application claims priority under 35 U.S.C. § 119 to German Patent Application No. 10 2024 206 133.6, filed Jul. 1, 2024, the entire contents of which are incorporated herein by reference.

### FIELD

One or more example embodiments relates to a method for selecting individual components of a medical imaging apparatus by selecting a group of individual components from a component set by way of an algorithm for machine learning. One or more example embodiments further relates to a method for training such an algorithm.

Independent of the grammatical term usage, individuals with male, female or other gender identities are included within the term.

### RELATED ART

Medical imaging apparatuses generally consist of a large number of individual components which can influence the image quality. Such imaging apparatuses can be, for example, a CT (computed tomography) device, an MRI (magnetic resonance imaging) device, an angiography device, an ultrasonic device and suchlike. Individual components would then be, for example, a mechanical and/or electrical component of the respective device. Mechanical components would be, for example, drives, bearings, slip rings and suchlike which can also influence the image quality. Furthermore, the imaging apparatuses typically also have signal transmitting components which can affect the image quality, particularly in respect of stability. A detector per se, which has, for example, an array of individual detectors (detector modules) can also be understood to be an imaging apparatus. In this case, the individual detector modules of, for instance, a CT detector which possibly have a photodiode and a collimator can be regarded as individual components.

For the construction of the imaging apparatus, the assembler selects the individual components that are needed from the inventory stock. All the individual components form a component set. Each individual component possesses specific properties that arise in the context of the production tolerances and measurement tolerances and result in characteristic dimensions, signal shapes and suchlike. Therefore, two essentially identically constructed imaging apparatuses are never identical, in particular with regard to the image quality achieved.

In the following, what is considered is mostly the example of a CT detector with its detector modules to be selected as individual components. However, example embodiments, in principle, are applicable to any desired other imaging apparatuses and/or components of a medical imaging apparatus.

CT detectors are constructed from a plurality of subcomponents, the so-called detector modules (e.g. photodiodes with a collimator). Since not all module combinations deliver the same image quality, but rather this depends heavily upon which modules they are constructed from and at which detector position (detector slot), it is necessary to construct the detectors in a sorted, and ideally an optimized, manner. However, there are very many possibilities for doing this. If, for example, given a typical inventory stock of 200 modules, it is desired to construct a detector from 40 modules, there results a total number of combinations in the order of magnitude of the number of atoms in the entire universe.

### SUMMARY

One or more example embodiments simplifies the selection of individual components of a medical imaging apparatus.

This is achieved with a method and an apparatus according to the independent claims. Advantageous developments are disclosed in the dependent claims.

### BRIEF DESCRIPTION OF THE DRAWINGS

Example embodiments will now be described in greater detail making reference to the accompanying drawings, in which:

FIG. 1 shows a view of an exemplary C-arm device;

FIG. 2 shows a schematic view of the functioning of an exemplary embodiment of a selecting and/or sorting method; and

FIG. 3 shows a block diagram of an exemplary embodiment of a training method.

### DETAILED DESCRIPTION

Constructing the detectors in a sorted, and ideally an optimized, manner can be addressed nowadays as a machine learning problem. Algorithms for machine learning (subsequently also referred to as AI: artificial intelligence) can be accessed, in particular, on neural networks, similarly to large language models, to generate sortings. Not only does this have the advantage that neural networks do this very efficiently and rapidly, but they can also be quantitatively optimized on the basis of different metrics.

A disadvantage thereof, however, is that changes in the sorting requirements of an AI can only be taken into account if it is retrained or is finely adjusted to the new conditions. The parameters must be adapted such that it can adapt to the new requirements.

This is a non-negligible problem. It can occur that, given difficult existing inventory stocks, a sorting AI does not return a useful sorting solution. Particularly in these scenarios, however, in the production process there is mostly a dependency upon the sorting algorithm. If now a new sorting can only be obtained in that the AI is retrained or sorting is carried out manually, this is associated with delays in the checking process.

Currently, so-called status values are also associated with the detector modules in addition to their measurement values. This takes place in the context of model checking and by way of these status values, the modules are distributed into different categories. There are therefore categories for modules which correspond to the standard requirements and categories for modules with particular properties or anomalies. Via the so-called status filter, rules can then be defined as to which categories are permitted for which detector slot. For example, a restriction can therefore be set that in the central region of the detector, only modules from a category without anomalies should be installed, since there the CT imaging reacts particularly sensitively to irregularities. For example, in the detector center, only detector modules from a particular manufacturer can be used, whereas at the detector edge, detector modules from another manufacturer can be used. A typical status value would therefore be, for example, the name of a supplier or the specification of a checker, that the relevant module can only be used at the edge since it does not decay rapidly or its collimator is dimensioned larger than average.

In addition, rules are parameterized using limit values for the measurement values and various comparison modes, which are used to compare the different modules with each other. For example, for each module, a mean value and/or a stability of the photon count rates is measured and recorded as a corresponding measurement value. Thus, limit values are specified and/or observed in relation to noise or instability.

It is subsequently checked whether the selected and/or sorted combination as a detector can achieve a desired image quality. Both the limit values in respect of the modules and also the sorting filter can be adapted by experts and technologists if necessary in order, for example, to improve the quality of the sorted detectors or to test what influence particular modules have at different detector slots.

On sorting by an AI, these limit values fall away and are still present only in an implied form: the neural network carries out these comparisons in a similar form in its calculations but it has determined and/or learned the applied limits during training. The status values can also be transferred to the network as input data, but they do not include any special significance aside from the measurement data. There is therefore a priori no possibility to trim the AI to observe desired conditions-other than via renewed training.

Li, Z., Yang, Z. and Wang, M. describe in “Reinforcement Learning with Human Feedback: Learning Dynamic Choices via Pessimism”, 2023. doi: 10.48550/arXiv.2305.18438, aspects of reinforcement learning from human feedback (RLHE).

Rafailov, R., Sharma, A., Mitchell, E., Ermon, S., Manning, C. D., and Finn, C. describe in “Direct Preference Optimization: Your Language Model is Secretly a Reward Model”, 2023. doi: 10.48550/arXiv.2305.18290, aspects of the direct preference optimization (DPO).

According to one or more example embodiments, therefore, a method is provided for selecting individual components of a medical imaging apparatus. This imaging apparatus can be, for example, a CT (computed tomography) device, an MRI (magnetic resonance imaging) device, an angiography device, an ultrasonic device and suchlike. A detector per se, which has, for example, an array of individual detectors can also be used here as an imaging apparatus. As described above, the individual components can be mechanical, electrical and, in particular, converter components. In particular, the individual modules of, for instance, a CT detector which possibly have a photodiode and a collimator can be regarded as individual components.

As a substantial step of the method according to one or more example embodiments, a selection of a group of individual components from a component set takes place by way of an algorithm for machine learning. Thus a plurality of individual components are selected from a component set. For example, from an inventory stock that is represented by the component set, 40 detector modules are selected as individual components. The component set is typically specified. In particular, it can be variable since the inventory stock changes, for example, with each removed component or newly added component.

The selection takes place by way of an algorithm for machine learning. An algorithm of this type can be realized by way of a linear regression algorithm, a support vector machine, an artificial neural network and suchlike. In general, the machine learning can be based upon so-called deep learning.

In a further step of the method according to one or more example embodiments, a provision and/or generation of a text input, which possibly describes one or more masks, takes place. The text input is preferably based upon natural language and can represent a so-called prompt. The prompt is thus an instruction signal or an input which is directed to the AI system, in particular, the algorithm for machine learning, in order to initiate a particular response or action. The text input can thus be, for example, the text: “Sort the detector modules so that the most stable modules are arranged in the center”.

The text input can describe a mask (or a plurality thereof) that is applied for selecting the individual components. The mask is then, in practice, integrated into the text input and the algorithm for machine learning is able to extract the mask from the text input and apply it.

In a subsequent step, therefore, the text input (e.g. the mask) is used in a selection of the group of individual components of the algorithm for machine learning. It is therefore simple for the user to input a corresponding text input for the selection and/or sorting process. In particular, he can describe, for example, the mask to be input during input in natural language. The mask extracted from the text input is therefore utilized as a filter for the selection and/or sorting process.

In an exemplary embodiment, it is provided that for each individual component of the component set, a measurement value and a property information item are present and the respective measurement values and property information items are used in the selection of the group of individual components by the algorithm for machine learning. Each individual component of the component set is thus described here by way of at least one measurement value and at least one property information item. The measurement value can be, for example, a variable mentioned in the introduction such as, for instance, stability, noise and suchlike. The property information item is, for example, a module label. This can be a particular class of component, a particular anomaly (e.g. checker remark: “To be used at the edge”) or a quality measure. For the selection of the individual components, three variables can therefore be made use of: The measurement value, the property information item (module label) and the mask of the text input. Thus, selection processes can be carried out reliably and easily in a targeted manner.

In a further exemplary embodiment, the selection includes a sorting in which a placement location is assigned to each selected individual component. Thus, an individual component is not only selected from the component set, but a placement location is also assigned to it in a targeted manner. The placement location can be, for example, a detector slot, that is, a position of a module in a detector. The sorting is necessary since it is less disturbing, for example, at the edge of the image if a detector module has a high level of noise. Therefore, if the detector modules are sorted so that those with low noise are arranged in the center of the detector, then there is an enhanced image quality in the image center which is of primary interest. This sorting problem arises for all the components of the imaging apparatus that are contained multiple times in the imaging apparatus.

According to a further exemplary embodiment, it is provided that the individual components are each detector elements of a position-sensitive detector. This has already been indicated by the above examples. Thus, for example, the whole detector detects an array of detector elements and/or detector modules and each detector element detects at its individual position. In this way, for example, electromagnetic rays or sound waves can be detected at specific positions. From the relevant position-dependent detector signals, corresponding images can be generated in two-dimensional resolution. If a plurality of recordings are obtained at different detector positions, then three-dimensional images can also be generated from the detector signals obtained.

In a special exemplary embodiment, it is provided that the position-sensitive detector is a CT detector. For example, such CT detectors 40 have detector modules which must be selected from a large number of detector modules in an inventory stock. In principle, the individual selected modules are often of the same type, although they differ in fine details that can be significant for the image quality of the entire detector. To this extent, a simple and rapid sorting which leads to a high image quality is of great significance. The aim of the sorting can however also be not only to produce a single detector with the highest quality from the inventory stock, but rather a plurality of detectors, each having high quality. To this extent, as the input variable for the algorithm for machine learning, the entire inventory stock and a corresponding sorting specification in relation to a plurality of detectors as text input can be relevant.

According to a further embodiment, the text input contains a system prompt with which it is specified in language for which system the group of individual components is selected. With the system prompt, it can therefore be specified very easily for which type or which types of imaging apparatus the individual components should be selected. For example, it can be stated that individual components are to be assembled for the detector of a special C-arm system. From this, the algorithm recognizes which specific requirements are directed, for example, to the detector modules.

In a further exemplary embodiment, it is provided that the text input contains a specialization prompt with which it is specified in language how a previously performed selection of individual components can be changed. With a specialization prompt of this type, for example, it can be specified that in the center of the detector more detector modules are inserted which show a low level of noise if this specialization has not taken place in the preceding sortings. Typically, the attempt is made with the specialization to achieve, for example, local improvements to the detector. In principle, however, a worsening can also be striven for with the specialization prompt if, for example, the quality of the detector modules is high enough in the center such that the images obtained therefrom have a higher quality than is necessary for the intended purpose. In this case, it could be specified with the specialization prompt that the quality of the individual modules can be somewhat lower on average in the center so that, for example, with the inventory stock, more detectors with adequate quality can be constructed.

According to one or more example embodiments, a method is also provided for training an algorithm for machine learning (AI unit, for example, with a neural network) for selecting individual components of a medical imaging apparatus. The method comprises the following steps:
Step S1: providing (and/or generating) a text input (e.g. a prompt which describes one or more masks),
Step S2: selecting a group of individual components from a (predetermined or variable) component set by way of the algorithm on the basis of the text input,
Step S3: obtaining a quality measure for an image quality of an image from the imaging apparatus constructed from the individual components selected according to step S2,
Step S4: generating a plurality of datasets by way of a plurality of repetitions of the step sequence S1 to S3 or S2 to S3, wherein each of the plurality of datasets contains an information item relating to the component set, an information item relating to the text input according to step S1, an information item relating to individual components selected according to step S2 (possibly including characterizing data and module labels) and the associated quality measure obtained according to step S3, and
Step S5: training the algorithm for machine learning with the datasets generated in step S4.

With regard to the algorithm for machine learning and the medical imaging apparatus, the statements above relating to the selection method also apply to the present training method.

The text input generated in step S1 is typically designated a prompt. The algorithm for machine learning which can include, for example, a large language model, disassembles the text input into tokens that it can further process. With the text input, one or more filters, i.e. one or more masks, are defined.

In step S2, a group of individual components is selected from the component set with the aid of the mask. As previously mentioned, the component set can be a predetermined set, for example, a current inventory stock. The component set can, in particular, also be variable if, for example, a typical inventory stock constantly changes. With the aid of the mask defined in step S1, the algorithm selects and/or sorts a group of individual components, for example, the detector modules for a CT detector.

In step S3, a quality measure is obtained for the image quality of an image which originates from an imaging apparatus the individual components of which have been selected according to step S2 and/or sorted. If the algorithm supplies, for example, a specific sorting of the detector modules, then a particular image can be obtained with the detector formed therefrom. This image possibly contains artifacts such as, for instance, rings, shadows and suchlike. Dependent upon the size of the artifacts, a corresponding quality results, which can be expressed with the quality measure.

According to step S4, a plurality of datasets are generated by repeating the aforementioned steps S1 to S3 multiple times. Where relevant, only the steps S2 to S3 are repeated multiple times and the text input generated in step S1 is used multiple times. The latter is useful, for example, if a plurality of detectors which meet the same conditions and/or text inputs are to be constructed from an inventory stock. The datasets obtained form a basic set for the training of the algorithm. Each dataset that is used for the training possesses an information item relating to the component set, i.e. the basic set and/or starting set from which the individual components are selected and possibly sorted. This information item relating to the basic set represents for the algorithm the starting point for the selection. In the simplest case, this information item relating to the component set represents only a binary information item that, for example, the component set is unchanged relative to that of another dataset. If required, however, this information item relating to the component set also contains the description of all the individual components of the component set. Furthermore, each dataset possesses an information item relating to the text input provided and/or generated in step S1. This text input which can be unchanged relative to other datasets, also represents a learning basis. Furthermore, each dataset possesses an information item relating to the individual components selected in the respective individual case according to step S2. Thus, in each dataset, a corresponding selection and/or sorting is documented. Furthermore, each dataset possesses a quality measure that belongs to the respective selection and was obtained in step S3. In this way, the correspondingly achieved image quality is also provided to the algorithm for each selection step for training.

This training of the algorithm for machine learning takes place in step S5 with the datasets generated in step S4. The algorithm is therefore specifically trained to select individual components from a component set with a simple text input such that a particular image quality is achieved. With this training method, therefore, an algorithm can be generated which, with a simple text input, provides for a desired image quality of a medical imaging apparatus.

According to a specific development, it is provided that the training according to step S5 takes place by way of reinforcement learning human feedback (RLHF) or direct preference optimization (DPO). Therefore, during the learning process, known approaches to learning can be accessed.

The training method just described with its variants can be used to generate and/or train the algorithm described above for selecting the individual components.

According to one or more example embodiments, an apparatus can also be provided with a computing facility (also referred to as a computing device) that is configured to carry out one of the above methods. According to this, if relevant, the computing facility can carry out the selection method and/or the training method.

Furthermore, an apparatus for producing an imaging apparatus with a production facility (also referred to as a production device) can be provided which has an algorithm for machine learning with which the aforementioned selection and/or sorting method can be carried out. With the production facility, the imaging apparatus and, in particular, a CT detector can be produced at least semi-automatically.

In addition, a computer program is provided comprising commands which, on execution of the program by a computer or the aforementioned apparatus, cause it to carry out one of the aforementioned methods.

In addition, a computer-readable storage medium is provided, comprising commands which, on execution by a computer or the aforementioned apparatus, cause it to carry out one of the aforementioned methods.

The exemplary embodiments set out in greater detail below represent preferred embodiments of the present invention.

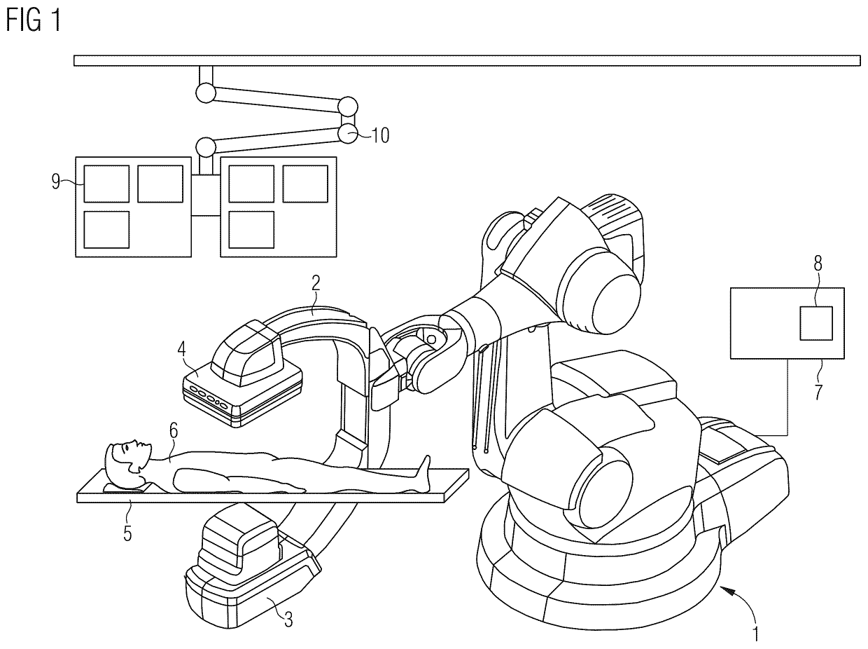

FIG. 1 shows by way of example a monoplanar X-ray system with a C-arm 2 held by a stand 1 in the form of a six-axis industrial or articulated arm robot at the respective ends of which an X-ray source, for example an X-ray radiator 3 with an X-ray tube and a collimator, and an X-ray image detector 4 as the image recording unit are mounted. The realization of the X-ray diagnostic facility (also referred to as an X-ray diagnostic device) is not dependent upon the industrial robot. Conventional C-arm devices can also be used.

Situated in the beam path of the X-ray radiator 3, on a tabletop 5 of a patient positioning table is a patient 6 to be examined or a technical object as the examination object. Attached to the X-ray diagnostic facility is a system control unit 7 with a computing facility 8 for image processing which receives and processes the image signals of the X-ray image detector 4 (for example, operating elements are not shown). The X-ray images can then be observed on displays of a monitor status indicator 9. The monitor status indicator 9 can be held via a ceiling-mounted, longitudinally movable, pivotable, rotatable and height-adjustable carrier system 10 with a jib arm and a carrier arm that is capable of being lowered.

One or more example embodiments extends the selecting and/or sorting procedure carried out by an AI with the aid, for example, of prompts in natural language. Similarly to inquiries in generative models such as stable diffusion, DALL-E, Midjourney or ChatGPT, thus desired requirements that can be made useful for the sorting process can be handed over to the AI.

An inquiry of this type can include, for example, a system prompt. A system prompt of this type represents an inquiry which relates to a particular system. For example, the same detector should always be produced. Thus, the system prompt can describe, for example, the detector category or the detector type. In this case, with respect to the individual detector modules, the algorithm will take note, for example, of particular properties in relation to the measurement values.

The inquiry can, however, also contain a specialization prompt. This prompt is a one-time inquiry which can be used individually to observe and take into account specific requirements. By way of example, a sorting has not met expectations and therefore a specific requirement is formulated that satisfies them.

In an inventory stock (general component set) there is, for example, a certain number of detector modules from which a group can be selected in order to be able to build up a detector. One or more module labels or characterizing data items and/or measurement values are associated with each detector module, i.e. each individual component. These module labels permit, apart from the characterizing data measured in the module checking, categorical information, such as, for example, classes of the modules or anomalies to be stored. These typically ensure that the modules can be used, but not without restrictions. Examples of module labels are, for instance, “anomalies in the collimator” (e.g. collimator very large or very small), “edge status” (module should be used at the detector edge due to, for example, a high noise level) or different module classes, established on the basis of decay times. If relevant, a plurality of module labels are associated with a detector module.

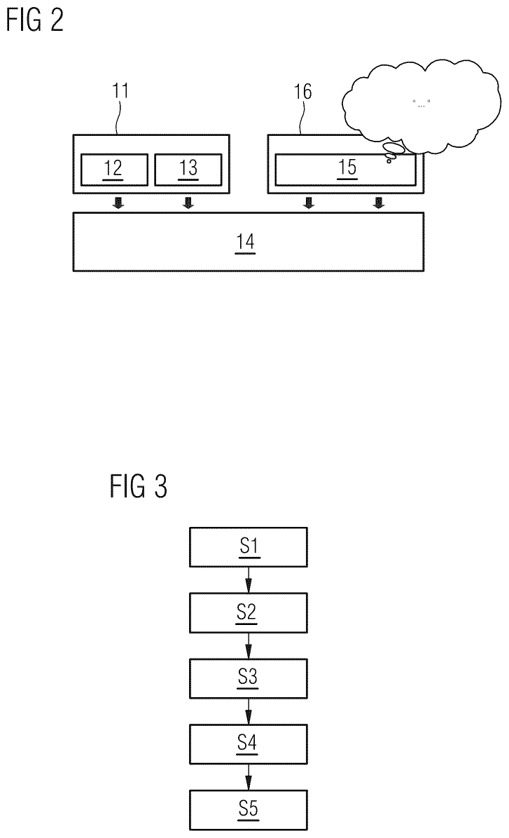

A detector has a large number of detector slots at each of which detector modules can be mounted. Now, masks can be defined which specify which module labels are permitted at which detector slots. Only detector modules that have corresponding module labels can then be selected for the relevant detector slots. Such masks offer the advantage that they are highly efficient and can naturally be integrated into the AI and/or the algorithm for machine learning in order to make use of the information of the labels. This is shown symbolically in FIG. 2. A component set 11, in this case a store content, comprises a large number of individual components (e.g. detector modules) each of which can be associated with characterizing data 12 and/or module labels 13. The characterizing data 12 is measurement values and/or checking values that have arisen during the checking of the individual components. For example, the characterizing data 12 can be a noise level or the variance of a photon counter rate. The module labels 13 can contain, for example, the aforementioned categorical information or anomalies.

This characterizing data 12 and the module labels 13 of the individual components from the component set 11 are fed to an algorithm for selecting and/or sorting for machine learning 14. This algorithm 14 can be a sorting algorithm. Thus the sorting algorithm 14 can select and/or sort a group of these individual components on the basis of the current store content 11 with the characterizing information of the individual components. In the case of the sorting, the sorting algorithm of a selected individual component indicates a fixed place, for example, a detector slot.

Apart from the input data from the existing store (characterizing data 12 and module label 13), a prompt 15 with a selecting and/or sorting inquiry can be passed to the algorithm 14. This prompt 15 can be freely formulated via an input interface or taken from a sorting specification 16 with predetermined prompts. For example, the prompt is: “Create a sorting in which the center has only XY modules, but the difference from the neighbor is not more than . . . ”. As is evident from the example, the prompt is not restricted to defining a single selecting and/or sorting condition. Rather, with a prompt, a plurality of conditions and/or combinations of conditions can preferably also be defined in natural language. The algorithm 14 converts the prompt 15, that is, the inquiry, into a context vector which can be used during sorting in order to make a suitable selection of individual components and/or modules.

This AI unit shown in FIG. 2 and/or the corresponding algorithm 14 can be optimized iteratively by training. This takes place, for example, in the five steps S1 to S5 which are shown in FIG. 3. Therein, the steps S1 and S2 can also be swapped or carried out in parallel with one another.

In step S1, a prompt 15, i.e. a text input, is generated or provided. The prompt 15 can be already generated and provided in a memory store. Alternatively, the provision of the prompt 15 can also take place via an input interface that an operator uses (e.g. a keyboard).

In step S2, the algorithm selects a group of individual components (e.g. 40 detector modules for a detector) from a component set 11 (e.g. store content) on the basis of the text input, i.e. the prompt 15. The text input can effectively describe a mask with which the sorting or selection must take place.

In step S3, a quality measure is obtained for the image quality of an image from the imaging apparatus which has been constructed according to step S2 from the selected individual components. The quality measure forms the result of the selection quantitatively. The quality measure can thus serve as feedback for the algorithm to be trained.

In step S4, a plurality of datasets are generated by

repeating the step sequence S1 to S3 or S2 to S3 multiple times. In the present sorting example, this means that a plurality of detectors with differently sorted detector modules are produced and checked with regard to image quality. Therein, the text input can be the same or different. The datasets for the training contain all the information necessary for this, such as for example, the existing inventory stock (including characterizing data and module labels of the individual components), the prompts input, the selected detector modules and/or individual components and the respective quality measure.

In step S5, the algorithm for machine learning 14 is trained with the datasets generated in step S4. This can be achieved, for example, via a reinforcement learning from human feedback (RLHF) or a direct preference optimization (DPO) approach. The AI is therefore optimized to use the prompt 15. The data needed for this can be created both in the production process and also separately.

A particular advantage of the solution according to one or more example embodiments as compared with known approaches consists therein that for the sorting and/or selection, conditions can also be used that are based upon measurement data. Furthermore, it is also possible with the individual components identified with labels, in the selection and/or sorting, to observe not only an individual component per se, but also subgroups of individual components, which together form the group to be selected. There is therefore, for example, no restriction as in the prior art, to consider each detector slot individually and to make only a choice of whether a module is suitable for a slot. Rather, with the method according to one or more example embodiments, sortings can also be achieved in which assessments are made across a plurality of slots.

A prompting via natural language further permits conditions to be introduced in the desired complexity, even without renewed training of the algorithm. This saves time in the production and makes it easier for experts and process technologies to formulate the requirements which they wish to set for the sorting and which possibly extend beyond the associations learned by the algorithm. This permits a suitable interplay of the algorithm with the staff members in practice. The experts can thus contribute their domain knowledge regarding good sortings and, simultaneously, the AI can recognize and take advantage of associations between the modules in the datasets to be used for training that extend beyond the existing domain knowledge.

By way of the proposed training, furthermore, over time, the association between language and the module properties can be learned in ever finer detail.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of transferred, being stored, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing facility) or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been described in accordance with preferred embodiments, it is obvious for the person skilled in the art that modifications are possible in all embodiments.

1. A method for selecting individual components of a medical imaging apparatus, the method comprising:
providing a text input; and
selecting a group of individual components from a component set using a machine learning algorithm and using the text input for the selection of the group of individual components by the machine learning algorithm.

2. The method of claim 1, wherein for each individual component of the component set, a measurement value and a property information item are present and the selecting uses the respective measurement values and property information items are used in the selection of the group of individual components by the machine learning algorithm.

3. The method of claim 1, wherein the selecting includes sorting based on a placement location assigned to each selected individual component.

4. The method of claim 3, wherein the individual components are each detector elements of a position-sensitive detector.

5. The method of claim 4, wherein the position-sensitive detector is a computed tomography (CT) detector.

6. The method of claim 1, wherein the text input contains a system prompt in a language, wherein the selecting selects for the selecting selects the group of individual components using the language.

7. The method of claim 1, wherein the text input contains a specialization prompt in a language regarding how a previously performed selection of individual components can be changed.

8. A method for training a machine learning algorithm for selecting individual components of a medical imaging apparatus, the method comprising:
providing a text input;
selecting a group of individual components from a component set using the machine training algorithm based on the text input;
obtaining a quality measure for an image quality of an image from the imaging apparatus constructed from the selected individual components;
generating a plurality of datasets by way of a plurality of repetitions of the providing, selecting and obtaining or the selecting and obtaining, wherein each of the plurality of datasets contains an information item relating to the component set, an information item relating to the text input, an information item relating to the selected individual components from the selecting and an associated obtained quality measure; and
training the machine learning algorithm with the plurality of datasets.

9. The method of claim 8, wherein the training includes reinforcement learning human feedback or direct preference optimization.

10. The method of claim 1, wherein the machine learning algorithm is trained by,
providing a training text input,
selecting a training group of individual components from a training component using the machine training algorithm based on the text input,
obtaining a training quality measure for a training image quality of a training image from the imaging apparatus constructed from the selected training individual components,
generating a plurality of training datasets by way of a plurality of repetitions of the providing, selecting and obtaining or the selecting and obtaining, wherein each of the plurality of training datasets contains an information item relating to the training component set, an information item relating to the training text input, an information item relating to the selected training individual components from the selecting and an associated obtained quality measure, and
training the machine learning algorithm with the plurality of training datasets.

11. An apparatus comprising:
a computing facility configured to cause the apparatus to perform the method of claim 1.

12. An apparatus for producing an imaging apparatus, the apparatus comprising:
a production facility including a machine learning algorithm configured to perform the method of claim 1.

13. A non-transitory computer-readable storage medium comprising commands which, when executed by an apparatus, cause the apparatus to perform the method of claim 1.

14. The method of claim 2, wherein the selecting includes sorting based on a placement location assigned to each selected individual component.

15. The method of claim 5, wherein the text input contains a system prompt in a language, wherein the selecting selects for the selecting selects the group of individual components using the language.

16. The method of claim 15, wherein the text input contains a specialization prompt in a language regarding how a previously performed selection of individual components can be changed.
